(19) United States
(12) Patent Application Publication
(10) Pub. No.: US 2008/0146990 A1
(43) Pub. Date: Jun. 19, 2008

(54) STROKE INTERVENTION APPARATUS AND METHOD

(75) Inventors: Mark L. Jenson, Greenfield, MN (US); William J. Drasler, Minnetonka, MN (US); Jagjit S. Gill, Rochester, MN (US)

Correspondence Address: VISTA IP LAW GROUP LLP, 12930 Saratoga Avenue, Suite D-2, Saratoga, CA, 95070, US

(73) Assignee: BOSTON SCIENTIFIC SCIMED, INC., Maple Grove, MN (US)

(21) Appl. No.: 11/956,734

(22) Filed: Dec. 14, 2007

Related U.S. Application Data
(60) Provisional application No. 60/870,364, filed on Dec. 15, 2006.

(51) Int. Cl. A61M 1/00

(52) U.S. Cl. 604/28

(57) ABSTRACT

Disclosed herein is an intrathecal catheter having a lumen adapted to transfer liquid between the proximal end of the catheter and its distal end; a pressure detector; and a first sealer, adapted to seal the catheter against a tissue. Also disclosed herein is a stroke intervention system comprising an intrathecal catheter, an infusate supply chamber in fluid communication with the catheter; a pump in fluid communication with the catheter; and a pressure gauge in fluid communication with the pressure detector. Disclosed are also methods of reducing neural injury in a patient during a stroke comprising introducing a catheter intravenously or into the cerebrospinal fluid (CSF) system of the patient; measuring a baseline pressure; infusing and withdrawing infusate fluid into the cerebral venous vasculature or the CSF system at regular intervals.

### RELATED APPLICATION DATA

The present application claims the benefit under 35 U.S.C. §119 to U.S. provisional patent application Ser. No. 60/870,364, filed Dec. 15, 2006. The foregoing application is hereby incorporated by reference into the present application in its entirety.

### FIELD OF THE INVENTION

The present invention is in the field of devices and methods to reduce neural injury during a stroke.

### BACKGROUND

The central nervous system (CNS) tissue is similar in many respects to tissue in other regions of the body. However, there are some significant and important differences which affect how the tissue responds to various diseases, injuries, and conditions. One such significant difference involves the extracellular fluid. The CNS tissue is serviced by arteries, veins, and capillaries, as are other tissues of the body. In most tissues, capillaries easily pass fluid, proteins and other materials in and out through the capillary walls between the blood and the extracellular fluid space. The fluid movement adds convective transport to supply cells with needed nutrients and chemicals, and to remove chemicals, materials, and unwanted waste products. It is this extracellular fluid that collects as lymph and passes through the lymph system. In CNS tissue, however, most capillaries are more selective and pass only certain substances between the blood and the extracellular fluid space. In most CNS tissue, the capillaries have tight junctions between cells which limits passive transport between the blood space and the extracellular space. Active transport of particular substances can take place; passage of larger protein molecules is more limited than smaller molecules.

Specialized tissues in particular regions of the CNS (choroids plexus) have capillaries that provide the majority of the fluid transport from the blood into the extracellular space. This fluid forms the cerebrospinal fluid (CSF) which surrounds the CNS structures and permeates CNS tissues. The CNS comprises four ventricles, which are filled with CSF, and certain passages allow CSF movement between the ventricles.

The typical CSF volume in a healthy person is approximately 150-250 mL, and the total production of CSF is approximately 0.35 mL/min. The rate of CSF absorption through the arachnoid villi into the veins is approximately the same as the rate of CSF production, such that in a normal person the volume of CSF remains relatively constant, although it can vary somewhat over time in response to conditions.

The choroids plexus tissues are typically associated with each of the four ventricles, and supply the ventricles with CSF by passage of fluid from the specialized capillaries in the choroids plexus tissues. Once in the ventricles, CSF follows to primary paths. The first path is from one ventricle to another, and into the subarachnoid space, which surrounds the CNS structures. The second primary path for CSF flow, which is from the ventricles, through the ventricle walls, into and throught he CNS tissues, provides for solute and material transport in the extracellular space around the cells of the CNS. This second path is not widely appreciated, but is important for the health and proper function of the brain and spinal cord.

The balance of nutrients, waste products, chemicals, solutes, and substances in the CSF is controlled by the capillaries in the choroids plexes. As the CSF passes around the cells of the CNS, the balance of materials passing into and out of the cells is also affected by the balance of materials in the CSF. In a stroke, the fluid and solute balance in regions undergoing or near an ischemic or hemorrhagic stroke can depart from normal, and the effects can include edema, pH imbalance, which is particularly detrimental to neural tissue, and exacerbation of ischemic insult.

In hemorrhagic stroke, including regions of an ischemic stroke, which have evolved and become hemorrhagic, blood leaks out through the blood vessel walls into the extracellular space. In an evolving stroke, this can be due tot the blood vessels becoming more “porous” and passing material more easily between cells. In a hemorrhagic stroke, this can be due to rupture of the blood vessel.

A known aid in the treatment of injuries and diseases elsewhere in the body is by affecting the flow of extracellular fluid and lymph which surrounds the tissues. For example, massage, particularly adapted to move fluid in desired directions is used to reduce swelling and speed healing. However, this is not practical in the CNS because the CNS tissue does not have a lymph system, and because the bones of the head and spine prevent conventional external massage.

### SUMMARY OF THE INVENTION

Disclosed herein is an intrathecal catheter having a proximal end and a distal end, the catheter comprising a first lumen adapted to transfer liquid between the proximal end and the distal end; a first aperture at the distal end of the first lumen, through which liquid enters or exits the lumen; a pressure detector; and a first sealer, adapted to seal the catheter against a tissue.

Also disclosed herein is a stroke intervention system comprising an intrathecal catheter, an infusate supply chamber in fluid communication with the catheter; a pump in fluid communication with the catheter; and a pressure gauge in fluid communication with the pressure detector.

Also disclosed herein is a method of reducing neural injury in a patient during a stroke comprising introducing a catheter into the cerebrospinal fluid (CSF) system of the patient; measuring a baseline pressure of the CSF system; infusing and withdrawing infusate fluid into the CSF system at regular intervals having a pulse frequency of between about 0.01 Hz to about 100 Hz, wherein the mean pressure of the CSF system during a pulse cycle is approximately the same as the measured baseline pressure of the CSF system.

Also disclosed herein is a method of reducing neural injury in a patient during a stroke comprising introducing a catheter intravenously into the patient; advancing the catheter to a location in the cerebral venous vasculature of the patient substantially adjacent to the site of the stroke; measuring a baseline venous pressure; infusing and withdrawing infusate fluid into a vein at regular intervals having a pulse frequency of between about 0.01 Hz to about 100 Hz, wherein the mean pressure of the vein during a pulse cycle is approximately the same as the measured baseline vein pressure.

### BRIEF DESCRIPTION OF THE DRAWINGS

It will be appreciated that the drawings are not necessarily to scale, with emphasis instead being placed on illustrating the various aspects and features of embodiments of the invention, in which:

FIG. 1A is an illustration of an embodiment of the catheter disclosed herein, having one lumen and one sealer.

FIG. 1B is an illustration of an embodiment of the catheter disclosed herein, having two lumens and one sealer.

FIG. 1C is an illustration of the cross-section of two lumens within a catheter, in which the two lumens are coaxial.

FIG. 1D is an illustration of the cross-section of two lumens within a catheter, in which one lumen is nestled within another lumen.

FIG. 1E is an illustration of the cross-section of two lumens within a catheter, in which the two lumens have circular cross-sections and are side-by-side.

FIG. 1F is another illustration of the cross-section of two lumens within a catheter, in which the two lumens have semicircular cross-sections and are side-by-side.

FIG. 1G is an illustration of an embodiment of the catheter disclosed herein, having two lumens and two sealers.

FIG. 2A is an illustration of an embodiment of the system disclosed herein having an infusate fluid tank.

FIG. 2B is an illustration of an embodiment of the system disclosed herein having an infusate fluid tank as well as a fluid receptacle.

### DETAILED DESCRIPTION OF THE ILLUSTRATED EMBODIMENTS

Embodiments of the present invention are described below. It is, however, expressly noted that the present invention is not limited to these embodiments, but rather the intention is that modifications that are apparent to the person skilled in the art and equivalents thereof are also included.

Disclosed herein are apparatus and methods for slow pulsing of CSF to effect a “massage” of portions of the CNS tissue. The pulsing of CSF affects the CSF imbalances and utilizes CSF to improve the extracellular environment and minimizes neurological injury and speeds recovery after a stroke.

In one aspect, disclosed herein is an intrathecal catheter having a proximal end and a distal end, the catheter comprising a first lumen adapted to transfer liquid between the proximal end and the distal end; a first aperture at the distal end of the first lumen, through which liquid enters or exits the lumen; a pressure detector; and a first sealer, adapted to seal the catheter against a tissue.

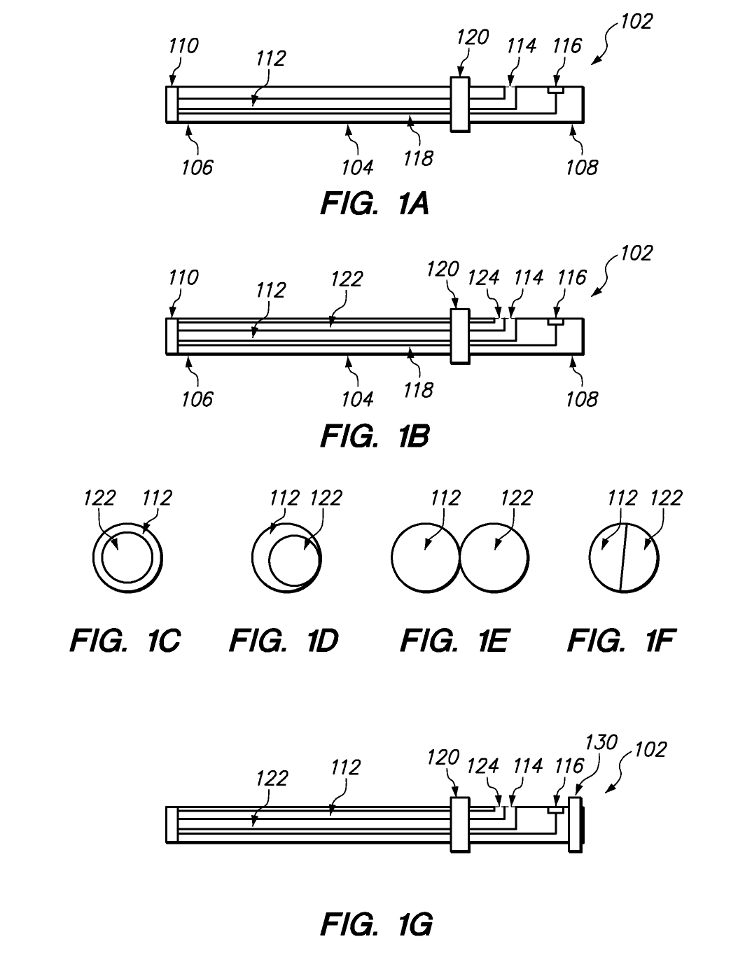

FIG. 1A shows one embodiment of the catheter disclosed herein. The catheter 102 comprises a body 104, having a proximal end 106 and a distal end 108. As used herein, “proximal” refers to the part of the device closest to the user, e.g., a physician. “Distal” refers to the end inserted into the patient, farthest away from the user. As with any specific component of the device, “proximal” refers to the part of the component closest to the proximal end of the device, whereas “distal” refers to the part of the component closest to the distal end of the device.

The embodiment of the catheter 102 shown in FIG. 1A also comprises a connector 110 at the proximal end. The connector 110 allows for the catheter 102 to be connected to other devices, such as a pressure gauge, a pump, an infusate supply, or a fluid receptacle, as discussed in detail below.

In some embodiments, the catheter 102 further comprises a lumen 112. The lumen 112 terminates at an aperture or opening 114 at the distal end of the catheter 102. The lumen 102 is configured to transport fluid from the proximal end 106 of the catheter 102 to the aperture 114 at the distal end 108 of the catheter 102.

In some embodiments, such as the one shown in FIG. 1A, the catheter 102 comprises only one lumen 112 having one aperture 114. In these embodiments, the lumen 112 transports fluid from a reservoir, such as an infusate supply, described below, located at the proximal end of the catheter 102, to the aperture 114 at the distal end of the catheter 102, and also transport fluid from the aperture 114 to a reservoir, such as a fluid receptacle, described below, at the proximal end of the catheter 102.

In other embodiments, such as the one shown in FIG. 1B, the catheter 102 comprises two lumens 112 and 122. Lumen 112 terminates at aperture 114, whereas lumen 122 terminates at aperture 124. In these embodiments, one lumen, e.g., lumen 112, transports fluid from a reservoir, such as an infusate supply, described below, located at the proximal end of the catheter 102, to the aperture 114 at the distal end of the catheter 102. Lumen 122, on the other hand transport fluid from the aperture 124 to a reservoir, such as a fluid receptacle, described below, at the proximal end of the catheter 102.

FIGS. 1C-1F show the cross-sections of some embodiments of the lumens 112 and 122. In some embodiments, such as the ones shown in FIGS. 1C and 1D, one of lumens 112 and 122 is nestled within the other of lumens 112 and 122. In some of these embodiments, e.g., FIG. 1C, the two lumens are coaxial, whereas in other embodiments, e.g., FIG. 1D, the wall of one lumen is adjacent to the wall of the other lumen. In other embodiments, such as the ones shown in FIGS. 1E and 1F, the lumens 112 and 122 are located side-by-side. While the figures show the cross-section of the lumens 112 and 122 to be circular, it is understood that the cross-section of the lumens can be any shape, e.g., a square, a triangle, a polyhedron, or a semicircle.

The catheter 102 can also comprise a pressure detector or sensor 116, which is connected to a pressure gauge (not shown) by a wire 118. The wire 118 can provide power to the pressure detector 116 and/or relay data obtained by the detector 116 to the pressure gauge. In some embodiments, a plurality of wires connect the detector 116 to the pressure gauge.

The catheter 102 can also comprise a sealer 120, as shown in FIG. 1A. The sealer 120 is configured to seal the catheter 102 against the tissue into which it is inserted, e.g., the venous wall or dura mater, such that no fluid can pass from one side of the sealer 120 to the other side, except through the catheter 102. In some embodiments, the sealer 120 is a balloon, which can be inflated by gas, such as air, or by liquid, such as water or saline.

In some embodiments, such as the one shown in FIG. 1G, the catheter 102 comprises two sealers, 120 and 130. In some of these embodiments, one sealer, e.g., sealer 120, is at the proximal side of the apertures 114 and 124 and pressure detector 116 and the other sealer, e.g., sealer 130, is at the distal side of the apertures 114 and 124 and pressure detector 116. Thus, in these embodiments, when both sealers 120 and 130 are deployed, e.g., inflated, the section of space between sealer 120 and sealer 130 is isolated from other fluids, such as CSF or blood, and is not in fluid communication with any fluid further up from sealer 130 and further down from sealer 120. The section between sealer 120 and sealer 130 is only in fluid communication with other fluids through lumen 112 or lumen 122.

In some embodiments, the catheter 102 further comprises a thermocouple sensor at the distal end that can measure the temperature of the fluid in the patient. In other embodiments, the catheter 102 further comprises a pulse monitor that can measure the heart rate of the patient into which the catheter 102 is inserted. In yet other embodiments, the catheter 102 comprises a heart rate sensor, a pH sensor, a glucose sensor, an oxygen sensor, or a carbon dioxide sensor.

In another aspect, disclosed herein is a stroke intervention system comprising: an intrathecal catheter, as described above, an infusate supply chamber in fluid communication with the catheter; a pump in fluid communication with the catheter; and a pressure gauge in fluid communication with the pressure detector.

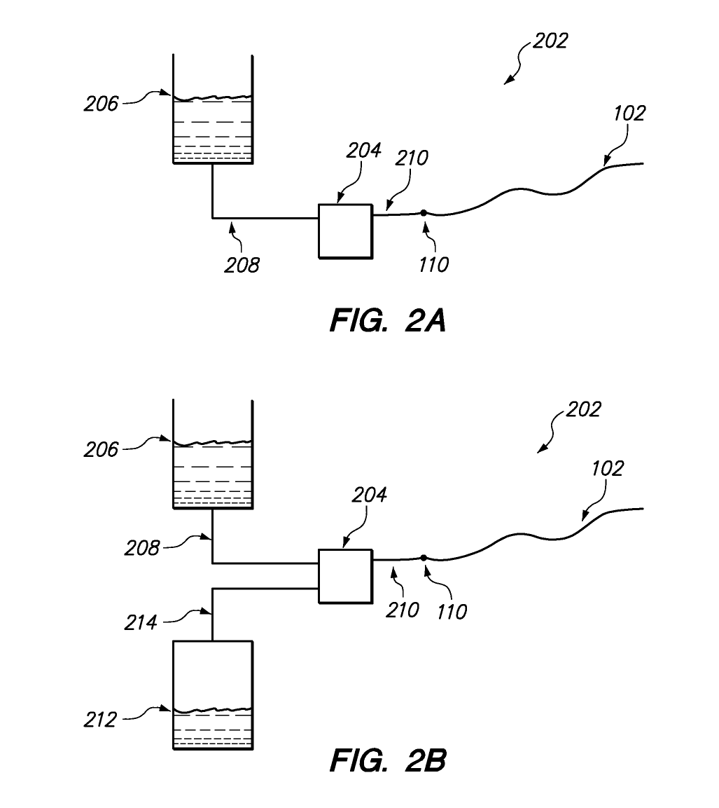

In some embodiments, such as the one shown in FIG. 2A, the stroke intervention system 202 comprises an intrathecal catheter 102, which has been described above. The catheter 102 is connected to the system 202 through the connector 110. The system 202 also comprises a pump mechanism 204 and an infusate supply tank 206. The infusate supply tank 206 is in fluid communication with the pump 204 through a line 208. The pump 204, in turn, is in fluid communication with a lumen 112 of the catheter 102 through a line 210.

In these embodiments, through its pumping action, the pump 204 draws fluid from the infusate supply tank 206 through the line 208 and transfers the fluid to the lumen 112 of the catheter 102 through the line 210. Fluid can then be dispensed within a patient's body through the aperture 114 of the lumen 112.

In some embodiments, the pump 204 can pump fluid forwards and backwards. In these embodiments, the pump 204 introduces fluid into the patient's body and a short time later, withdraws fluid from the patients body and returns the fluid to the tank 206.

In some embodiments, it is advantageous that the infusate fluid in the infusate supply tank not become contaminated with the fluid withdrawn from the patient's body. In some of these embodiments, such as the one shown in FIG. 2B, the system 202 further comprises a fluid receptacle 212, which is in fluid communication with the pump 204 through a line 214. In these embodiments, the catheter 102 comprises two lumens 112 and 122. The line 210 also comprises two lumens, each configured to attach to one of the lumens 112 and 122 through the connector 110. The pump 204 withdraws fluid from the tank 206 through the line 208 and transfers it to one of the lumens of the line 210, which is then pushed through one of lumens of the catheter 102, e.g., lumen 112, and out the aperture 114. The pump 204 then reverses direction at a short time later and withdraws fluid through the aperture 124 and lumen 122, whereupon the fluid is transferred to the receptacle 212 through line 214.

Pump 204 is configured such that it can cycle between introducing fluid and withdrawing fluid. In some embodiments, pump 204 is configured such that the user can determine the frequency with which fluid can be introduced into and withdrawn from a patient's body, i.e., the pulse frequency or the cycle frequency.

In some embodiments, the pulse frequency is between about 0.01 Hz to about 100 Hz. In other embodiments, the pulse frequency is between 0.1 Hz and 10 Hz. In some embodiments, the pulse pressure waveform is sinusoidal. In other embodiments, the waveform is not sinusoidal. In some embodiments the waveform is symmetric. In other embodiments, the waveform varies in shape, magnitude, and/or duration between the positive and negative portions of the waveform, or from one pulse to the next. The positive portion of the waveform refers to the pump action introducing fluid into the patient, whereas the negative portion of the waveform refers too the pump action withdrawing fluid from the patient.

In some embodiments, the system 202 comprises a pressure gauge (not shown). The pressure gauge is connected to the pressure detector 116 at the distal end of the catheter 102. The pressure gauge, in combination with the pressure detector 116, can determine the pressure of the medium into which the catheter 102 is inserted, e.g., the venous pressure or the CSF pressure.

In some embodiments, the pressure gauge measures the pressure of the medium into which the catheter 102 is introduced before any fluid is introduced thereto or withdrawn therefrom. This pressure measurement is a baseline measurement. It can represent the venous pressure or the pressure of the CSF. In some embodiments, the baseline measurement is the mean value of the pressure obtained over a given amount of time. Normally, in humans, CSF pressure is between 5 and 15 mmHg, while venous pressure is between 0-5 mmHg. However, many factors affect the pressure of either fluid system and it is preferred to measure the baseline pressure prior to introducing fluid into the system.

In some embodiments, the pump 204 introduces fluid into a patient such that the local pressure at the point where fluid is introduced becomes greater than the baseline pressure. The maximum pressure, or the high point, can be a pre-set value determined by the user. The pump 204 can also withdraw fluid from the patient such that the local pressure at the point where fluid was withdrawn drops below the baseline pressure. The minimum pressure, or the low point, can be a pre-set value determined by the user.

In some embodiments, the high point is between about 0 mmHg to about 20 mmHg greater than the baseline pressure. In some embodiments, the low point is between about 0 mmHg to about 20 mmHg lower than the baseline pressure. In other embodiments, the high point is between about 0 mmHg to about 40 mmHg greater than the baseline pressure. In some embodiments, the low point is between about 0 mmHg to about 40 mmHg lower than the baseline pressure.

In some embodiments, the system 202 comprises a heart rate monitor, which in combination with a heart rate sensor on the catheter 102, can measure the heart rate of the patient. In some of these embodiments, the pump 204 automatically adjusts its cycle frequency to match the heart rate of the patient.

The infusate fluid is a fluid that once introduced into the patient, does not cause damage or injury to the patient. In some embodiments, the infusate fluid is selected from the group of water, saline, native CSF, synthetic CSF, donor CSF, native blood, donor blood, synthetic blood substitute, blood fractions, an aqueous solution, and an aqueous suspension. By “native CSF” or “native blood” it is meant CSF or blood that was withdrawn previously from the patient. By “donor CSF” or “donor blood” it is meant CSF or blood that was withdrawn from another individual. By “synthetic CSF” or “synthetic blood substitute” it is meant a solution that comprises components not naturally found in CSF or blood, but the solution can be used to replace lost CSF or blood in an individual.

In another aspect, disclosed herein is a method of reducing neural injury in a patient during a stroke comprising introducing a catheter into a fluid system of the patient; measuring a baseline pressure of the fluid system; infusing and withdrawing an infusate fluid into the fluid system at regular intervals having a pulse frequency of between about 0.01 Hz to about 100 Hz, wherein the mean pressure of the fluid system during a pulse cycle is approximately the same as the measured baseline pressure of the fluid system.

In some embodiments, the fluid system is the cerebrospinal fluid (CSF) system and the catheter is introduced into the CSF system. In other embodiments, the fluid system is the venous blood, and the catheter is introduced intravenously.

In some of the embodiments where the catheter is introduced into the CSF system, the pressure of the CSF system during the pulse cycle is in the range of about ±20 mmHg from the measured baseline pressure. In some of the embodiments where the catheter is introduced intravenously, the venous pressure during the pulse cycle is in the range of about ±40 mmHg from the measured baseline venous pressure.

In some embodiments, the pulse frequency is between about 0.1 Hz to about 10 Hz. In other embodiments, the pulse frequency is an integer multiple of the heart rate of the patient. In some of these embodiments, the pulse frequency is substantially the same as the heart rate of the patient. In some of these embodiments, the pulse is in sync with the heart rate of the patient. In other embodiments, the pulse is out of phase with the heart rate of the patient. In some of these embodiments, the pulse is 180° out of phase with the heart rate of the patient.

In some embodiments, when the catheter 102 is introduced into the CSF system, the methods disclosed herein further comprise temporarily blocking off lateral or medial apertures, thereby isolating a portion of the CSF system for treatment. In other embodiments, when the catheter 102 is introduced intravenously, the methods disclosed herein further comprise temporarily blocking off a portion of the cerebral venous vasculature, thereby isolating a portion of the cerebral venous vasculature for treatment. In these embodiments, the physician inflates the balloons 120 and 130 on the catheter 102 such that the space between the two balloons is blocked off and is no longer in fluid communication with the rest of the fluid system, e.g., the CSF system or the venous blood. Introducing and withdrawing fluid locally will then have the effect of “massaging” a local portion of the CNS. In this manner, the therapeutic effect of the methods described herein are localized. Blocking the treatment portion also have the added advantage that edema to other areas of the CNS not affected by the stroke, which can be very dangerous, can also be controlled.

In certain embodiments, the catheter 102 is introduced into a cerebral ventricle and the infusate fluid is introduced and withdrawn from the ventricle. In some embodiments, the ventricle is the 4^{th} ventricle. In other embodiments, the catheter 102 is introduced into the central canal of the central nervous system and the infusate fluid is introduced and withdrawn from the central canal.

A number of medications are known in the art that are effective to treat stroke. These include, but are not limited to, antiplatelet agents (such as aspirin (acetylsalicylic acid), clopidogrel (Plavix®), dipyridamole (Aggrenox®, Persantine®), ticlopidine (Ticlid®)), anticoagulants (such as heparin (Calciparine®, Liquaemin®), warfarin (Coumadin®)), and thrombolytic agents (such as tPA (tissue plasminogen activator) (Activase®)). In some embodiments, the infusate fluid comprises a therapeutically effective amount of a medicament used in the treatment of stroke.

In some embodiments, it is effective to cool the fluid surrounding the central nervous system while treating the stroke. Therefore, in some embodiments, the infusate fluid is cooled to lower than room temperature prior to its infusion into the fluid system.

In some embodiments, the osmolarity of the infusate fluid is different than the osmolarity of the CSF fluid, whereby water from extracellular space and/or cellular space is drawn into the CSF fluid. In other embodiments, the osmolarity of the infusate fluid is different than the osmolarity of the venous blood, whereby water from the extracellular space and/or cellular space is drawn into the venous vasculature system.

It is understood by those of skilled in the art that the steps in the above method can be practiced in various different orders. The listing of the steps in the particular order described above does not, and should not, limit the disclosed method to the particular disclosed order of steps.

The invention may be embodied in other specific forms besides and beyond those described herein. The foregoing embodiments are therefore to be considered in all respects illustrative rather than limiting, and the scope of the invention is defined and limited only by the appended claims and their equivalents, rather than by the foregoing description.

1. An intrathecal catheter having a proximal end and a distal end, the catheter comprising
a first lumen adapted to transfer liquid between the proximal end and the distal end;
a first aperture at the distal end of the first lumen, through which liquid enters or exits the lumen;
a pressure detector; and
a first sealer, adapted to seal the catheter against a tissue.

2. The catheter of claim 1, further comprising a second lumen, wherein the first lumen is adapted to transfer liquid from the proximal end to the distal end, and the second lumen is adapted to transfer liquid from the distal end to the proximal end.

3. The catheter of claim 2, further comprising a second aperture at the distal end of the second lumen.

4. The catheter of claim 1, wherein the first sealer is a balloon.

5. The catheter of claim 1, wherein the tissue is selected from the group of venous wall and dura mater.

6. The catheter of claim 1, further comprising a second sealer, wherein the first aperture is located between the first sealer and the second sealer.

7. The catheter of claim 1, wherein the second sealer is a balloon.

8. A stroke intervention system comprising:
an intrathecal catheter, the catheter comprising
a first lumen adapted to transfer liquid between the proximal end and the distal end;
an aperture at the distal end of the lumen, through which liquid enters or exits the lumen;
a pressure detector; and
a first sealer, adapted to seal the catheter against a tissue;
an infusate supply chamber in fluid communication with the catheter;
a pump in fluid communication with the catheter; and
a pressure gauge in fluid communication with the pressure detector.

9. The system of claim 8, further comprising a fluid receptacle in fluid communication with the catheter.

10. The system of claim 8, wherein the catheter further comprises a second lumen, wherein the infusate supply is in fluid communication with the first lumen, the fluid receptacle is in fluid communication with the second lumen, and the pump is in fluid communication with both the first lumen and the second lumen.

11. The system of claim 10, wherein the pump is configured to cycle between introducing fluid through the first lumen in a first direction and withdrawing fluid through the second lumen in a second direction, wherein the second direction is the opposite of the first direction.

12. The system of claim 8, further comprising a frequency modulator configured to set the frequency with which the pump introduces and withdraws fluid through the first and second lumens.

13. The system of claim 8, further comprising a heart rate monitor.

14. A method of reducing neural injury in a patient during a stroke comprising:
introducing a catheter into a fluid system of the patient;
measuring a baseline pressure of the fluid system;
infusing and withdrawing an infusate fluid into the fluid system at regular intervals having a pulse frequency of between about 0.01 Hz to about 100 Hz, wherein the mean pressure of the fluid system during a pulse cycle is approximately the same as the measured baseline pressure of the fluid system.

15. The method of claim 14, wherein the pulse frequency is between about 0.1 Hz to about 10 Hz.

16. The method of claim 14, wherein the infusate fluid comprises a therapeutically effective amount of a medicament.

17. The method of claim 14, wherein the infusate fluid is cooled to lower than room temperature prior to its infusion into the fluid system.

18. The method of claim 14, wherein the infusate fluid is selected from the group consisting of a native blood, donor blood, synthetic blood substitute, blood fractions, an aqueous solution, an aqueous suspension, a native CSF, and a synthetic CSF.

19. The method of claim 14, wherein the pulse frequency is an integer multiple of the heart rate of the patient.

20. The method of claim 14, wherein the pulse frequency is substantially the same as the heart rate of the patient.

21. The method of claim 14, wherein the fluid system is the cerebrospinal fluid (CSF) system.

22. The method of claim 21, wherein the pressure of the CSF system during the pulse cycle is in the range of about ±20 mmHg from the measured baseline pressure.

23. The method of claim 21, further comprising temporarily blocking off lateral or medial apertures, thereby isolating a portion of the CSF system for treatment.

24. The method of claim 21, wherein the catheter is introduced into a cerebral ventricle and the infusate fluid is introduced and withdrawn from the ventricle.

25. The method of claim 21, wherein the catheter is introduced into the central canal of the central nervous system and the infusate fluid is introduced and withdrawn from the central canal.

26. The method of claim 21, wherein the osmolarity of the infusate fluid is different than the osmolarity of the CSF fluid, whereby water from extracellular space and/or cellular space is drawn into the CSF fluid.

27. The method of claim 14, wherein the fluid system is the venous blood.

28. The method of claim 27, wherein the venous pressure during the pulse cycle is in the range of about ±40 mmHg from the measured baseline venous pressure.

29. The method of claim 27, further comprising temporarily blocking off a portion of the cerebral venous vasculature, thereby isolating a portion of the cerebral venous vasculature for treatment.

30. The method of claim 27, wherein the osmolarity of the infusate fluid is different than the osmolarity of the venous blood, whereby water from the extracellular space and/or cellular space is drawn into the venous vasculature system.
